(19) United States
(12) Patent Application Publication
(10) Pub. No.: US 2025/0214066 A1
(43) Pub. Date: Jul. 3, 2025

(54) BLACK PHOSPHORUS-MODIFIED COPPER-BASED CATALYST AND NEW USE IN HYDROGENATION REDUCTION REACTION OF ORGANIC COMPOUNDS THEREOF

(71) Applicants: HUBEI XINGFA CHEMICALS GROUP CO, LTD, Yichang, Hubei (CN); HUBEI XINGFA PHOSPHORUS CHEMICALS RESEARCH INSTITUTE CO., LTD, Yichang, Hubei (CN)

(72) Inventors: Licheng BAI, Yichang, Hubei (CN); Min ZHOU, Yichang, Hubei (CN); Jiannan WANG, Yichang, Hubei (CN); Fanzhang ZENG, Yichang, Hubei (CN); Chuan XIE, Yichang, Hubei (CN); Xuefeng YU, Yichang, Hubei (CN); Chang LIU, Yichang, Hubei (CN)

(73) Assignees: HUBEI XINGFA CHEMICALS GROUP CO, LTD, Yichang, Hubei (CN); HUBEI XINGFA PHOSPHORUS CHEMICALS RESEARCH INSTITUTE CO., LTD, Yichang, Hubei (CN)

(21) Appl. No.: 18/686,975

(22) PCT Filed: Jun. 26, 2023
(86) PCT No.: PCT/CN2023/102394

§ 371 Date: Feb. 27, 2024

(30) Foreign Application Priority Data
Sep. 16, 2022 (CN) 202211131270.7

(51) Int. Cl. B01J 27/18; B01J 37/10; B01J 37/18

(52) U.S. Cl. CPC B01J 27/1817; B01J 37/10; B01J 37/18; B01J 2231/641

(57) ABSTRACT

A method for preparing a black phosphorus-modified copper-based catalyst for organic hydrogenation is provided. The method comprises: preparing the black phosphorus-modified copper-based catalyst by a hydrothermal, a stirring and a ball milling mode with a copper-based catalyst as the active component and black phosphorus as the auxiliary agent. The present invention greatly improves the catalytic performance of the copper-based catalyst while reducing the usage amount of the catalyst through the synergistic effect between black phosphorus and copper, which is beneficial to improving the economic benefits of industrial production. Finally, an energy-saving, environment-friendly and efficient organic hydrogenation catalytic process is achieved. The method of the present invention has the characteristics of high efficiency, simplicity, green and pollution-free reaction process and the like, so has high industrial application value.

### FIELD

The present invention relates to the technical field of catalytic hydrogenation, and discloses a method for preparing a black phosphorus-modified copper-based catalyst for organic hydrogenation.

### BACKGROUND

Organic compounds such as olefin and benzene are important chemical raw materials, and their hydrogenated products are widely used in industrial production. Taking styrene and dimethyl oxalate as examples, ethylbenzene, the product of selective hydrogenation of styrene, has good stability and plays an important role in gasoline production and arene industry. Ethylene glycol, the product of hydrogenation of dimethyl oxalate, is also an important organic product and raw material, which has a wide range of applications and growing market prospects. Therefore, organic hydrogenation plays an important role in the industrial field.

The active components of hydrogenation catalysts commonly used in hydrogenation reactions of organic compounds such as olefins and benzene in industry include Pd, Pt, Au and the like. The high cost and low abundance of noble metal catalysts greatly limit their large-scale application. Therefore, people turn to the development of non-noble metal catalysts. Copper is widely used in the field of catalysis because of its special electronic structure and high catalytic activity. Cu-based catalysts are ideal substitutes for noble metal catalysts because of their low cost and good durability. A copper-based catalyst for selective hydrogenation of acetylene is produced by methods such as heating reduction and heat treatment in the method of the patent CN 111437852 A. An efficient copper-based catalyst for hydrogenation of dimethyl oxalate to ethylene glycol is produced by the hydrothermal method and the plasma treatment method in the patent CN 111905734 A. The above two preparation methods have high hydrogenation activity and stability, but their reaction conditions are harsh and the preparation processes are complicated.

### SUMMARY

In order to make up for the shortcomings of the prior art, the present invention provides a method for preparing a black phosphorus-modified copper-based catalyst for organic hydrogenation. The present invention uses black phosphorus as a dopant to modify a copper catalyst. Black phosphorus, as a new two-dimensional material, has a corrugated honeycomb structure and contains lone pair electrons. According to theoretical calculation, the empty orbit of copper can interact with black phosphorus through a cation-x bond, thereby forming a stable structure independently. Through the synergistic effect of black phosphorus and copper, the catalytic activity of the catalyst can be effectively improved. This method has the advantages of simple processes and mild reaction conditions, and has a significant improvement effect on the conversion ratio of organic hydrogenation.

The present invention is implemented by the following technical schemes:

A black phosphorus-modified copper-based catalyst, wherein the copper-based catalyst is selected from one or more of the group consisting of elemental copper, copper oxide, cuprous oxide, and tetra-copper trioxide; and a mass fraction of the black phosphorus is 0.001 wt %-50 wt %.

A material of black phosphorus in the black phosphorus-modified copper-based catalyst is selected from one or more of the group consisting of black phosphorus powder, black phosphorus quantum dots, black phosphorus nanosheets and black phosphorus crystals; and the mass fraction of the black phosphorus is 0.01 wt %-50 wt %.

The raw material of black phosphorus in the black phosphorus-modified copper-based catalyst is selected from the black phosphorus crystals; and a mass fraction of the black phosphorus is selected from any one of the group consisting of 0.01 wt %, 0.02 wt %, 0.03 wt %, 0.04 wt %, 0.05 wt %, 0.06 wt %, 0.07 wt %, 0.08 wt %, 0.09 wt %, 0.10 wt %, 0.20 wt %, 1 wt %, 10 wt %, 20 wt %, 30 wt %, 40 wt %, and 50 wt %.

A method for preparing the black phosphorus-modified copper-based catalyst, including the following steps: (1) adding a raw material of black phosphorus into a copper-based catalyst to form a mixture; (2) allowing the mixture to react; and (3) further separating a product obtained after the reaction is completed, drying, roasting and reducing the product under hydrogen, to obtain the black phosphorus-modified copper-based catalyst.

The reaction in the step (2) is one or more of a hydrothermal reaction, a stirring reflux reaction, or ball milling.

In the hydrothermal reaction, the mixture formed by adding the raw material of black phosphorus into the copper-based catalyst is transferred to a polytetrafluoroethylene lining at a hydrothermal temperature of 120-200° C. for 1-8 h.

In the stirring reflux reaction, the mixture formed by adding the raw material of black phosphorus into the copper-based catalyst is transferred to a reactor, and reacted under reflux for 0.1-10 h at a temperature of 100-200° C. and a stirring speed of 50-1000 r/min under stirring.

In the ball milling, the mixture formed by adding the raw material of black phosphorus into the copper-based catalyst is ball milled for 1-4 h at a rotating speed of 800-1200 r/min. Wherein ball milling is carried out in a ball milling tank well known to those skilled in the art, wherein the material of the ball milling tank and grinding balls is any one or more of agate, zirconia, 304 stainless steel, polytetrafluoroethylene and polyurethane, a diameter of the grinding ball is 1-50 mm, and a mass ratio of the copper-based catalyst to the grinding ball is 1:1-1:1000.

During the hydrothermal reaction and the stirring reflux reaction, the raw material of black phosphorus is first dispersed in a solvent selected from one or more of the group consisting of ethanol, N-methylpyrrolidone, N-vinylpyrrolidone, N-cycloethylpyrrolidone, N-octylpyrrolidone, formamide, N-methylformamide, N,N-dimethylformamide, N,N-dimethylacetamide, dimethyl sulfoxide, methanol, ethanol, ethylene glycol, isopropanol, tert-butanol, acetone, 2-pentanone and water, to form a solution of black phosphorus.

During the ball milling, the raw material of black phosphorus is wetted by adding the solvent and then ball milled.

The solvent is the environment to achieve uniform dispersion of the raw material of black phosphorus in the solvent. In fact, black phosphorus does not dissolve in these solvents. As long as the purpose of dispersion is achieved, the solvent is within the protection scope of the present invention.

During the hydrothermal reaction, the stirring reflux reaction or the ball milling, a surfactant is selectively added, and the surfactant is selected from one or more of the group consisting of cetyltrimethyl ammonium bromide, cetyltrimethyl ammonium chloride, cetyltriethylammonium bromide, octadecyl dimethyl ammonium chloride, octadecyl trimethyl ammonium bromide, Pluronic F127, polyvinylpyrrolidone, sodium dodecyl sulfate, and sodium dodecyl benzene sulfonate, and has a mass fraction of 0.1 wt %-10 wt %, or further preferably, has a mass fraction of 0.1 wt %-5 wt %; or further preferably, the surfactant is selected from cetyltrimethyl ammonium bromide, and has a mass fraction selected from any one of the group consisting of 0.3 wt %, 0.5 wt %, 1.0 wt %, 1.5 wt %, 2.0 wt %, 2.5 wt %, 3.0 wt %, 3.5 wt %, 4.0 wt %, 4.5 wt %, and 5.0 wt %.

The surfactant is used to achieve the rapid and uniform dispersion of the raw material of black phosphorus and the copper-based raw material in the solvent. As long as the purpose of surface activation of the raw material of black phosphorus and the copper-based raw material and the purpose of better contact between the raw material of black phosphorus and the copper-based raw material are achieved, the surfactant is within the protection scope of the present invention.

Phytic acid is selectively added into a mixed solution before the hydrothermal reaction, the stirring reflux reaction or the ball milling, and stirred and mixed well while heating up to 80-100° C. under an oil bath.

The product obtained after the reaction in the step (3) is further separated, and the separation includes any one of centrifugation, sieving and filtration.

The centrifugation refers to a product obtained after the hydrothermal reaction or the stirring reflux reaction is centrifuged at a centrifugal rotation speed of 1000-10000 r/min, and then a product obtained is dried in an oven at 30° C.-100° C. for 6-20 h.

The sieving refers to a product obtained after ball milling is sieved to 800 meshes or more, and then a product obtained is dried in an oven at 30° C.-100° C. for 6-20 h. The filtration refers to a product obtained after the hydrothermal reaction or the stirring reflux reaction is filtered in a filter screen, and then dried in an oven at 30° C.-100° C. for 6-20 h.

The roasting has a temperature of 200-700° C., and an inert atmosphere used during the roasting is selected from any one of the group consisting of argon, nitrogen and helium.

The reduction under hydrogen refers to a reduction reaction conducted after heating up to 160-450° C. under a mixed atmosphere of hydrogen and nitrogen usually for 1-15 h. A product obtained after the reduction reaction is the black phosphorus-modified copper-based catalyst; and in the mixed atmosphere of hydrogen and nitrogen, a volume fraction of hydrogen is 5-10%.

Another technical scheme of the present invention is new use of the black phosphorus-modified copper-based catalyst in a hydrogenation reduction reaction of an organic compound, wherein the organic compound includes any one of styrene, phenylacetylene, quinoline, furfural, dimethyl oxalate, nitrobenzene, p-chloronitrobenzene, p-nitrophenol, and ethyl acetate.

The method of the black phosphorus-modified copper-based catalyst in a hydrogenation reduction reaction of an organic compound includes the following steps:

In a preferred scheme, a solution of an organic compound is transferred to a lining of an autoclave, into which is added a catalyst; hydrogen is introduced in a closed environment, and continuously introduced after air is replaced by hydrogen, which usually requires 3-5 cycles to replace the air completely; hydrogen is continuously introduced to control a pressure of 0.5-10 MPa and a temperature of 60° C.-200° C. in the reaction kettle; and the mixture is reacted for 0.5-5 h to obtain a reduction product.

In a preferred scheme, a mass ratio of the catalyst to the organic compound is 1:3-10; a solvent used in the solution of the organic compound is selected from the group consisting of C1-C4 alcohol solvents, and a mass concentration of the organic compound is 10-50%.

Preferably, the organic compound used is one of styrene, phenylacetylene, quinoline or dimethyl oxalate.

The present invention greatly improves the catalytic performance and catalytic stability of the copper-based catalyst while reducing the usage amount of the catalyst through the synergistic effect between black phosphorus and copper, which is beneficial to improving the economic benefits of industrial production. Finally, an energy-saving, environment-friendly and efficient organic hydrogenation catalytic process is achieved. The method of the present invention has the characteristics of high efficiency, simplicity, green and pollution-free reaction process and the like, so has high industrial application value.

### BRIEF DESCRIPTION OF THE DRAWING

FIG. 1 is an electron microscope image of 0.03 wt % BP-Cu-0.3 prepared in Example 1, with black phosphorus in white and tetra-copper trioxide in black.

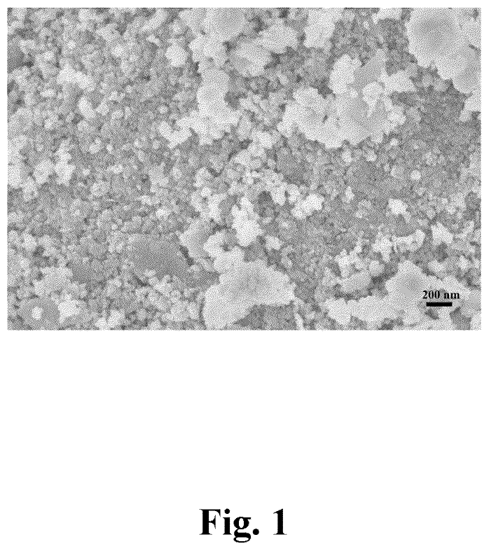

FIG. 2 is the XRD pattern of 0.03 wt % BP-Cu-0.3 prepared in Example 1 before reduction, and the main ingredient is tetra-copper trioxide. Because there is less black phosphorus, the diffraction peak of black phosphorus is not shown on the XRD.

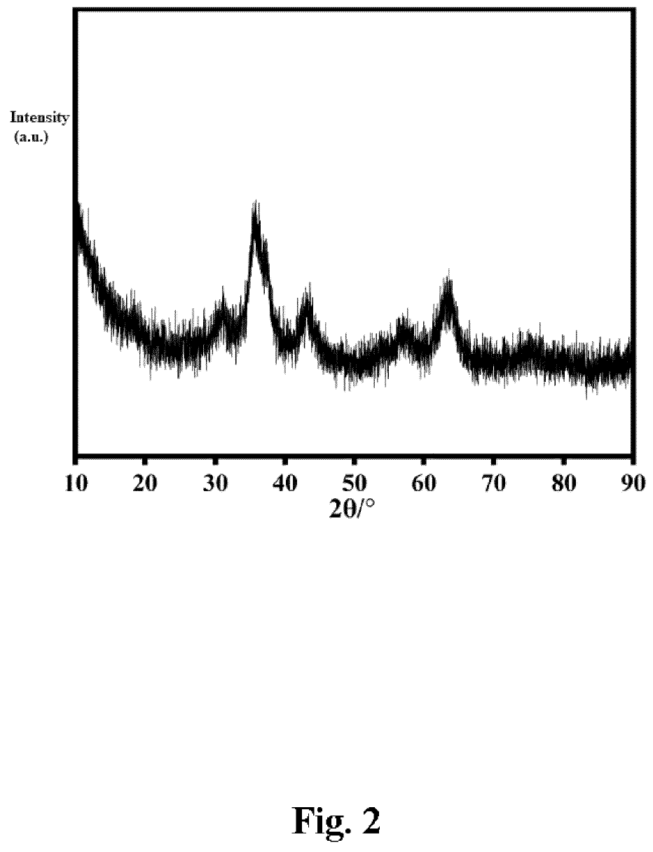

FIG. 3 is the EDS pattern of 0.03 wt % BP-Cu-0.3 prepared in Example 1, which confirms the existence of the element P.

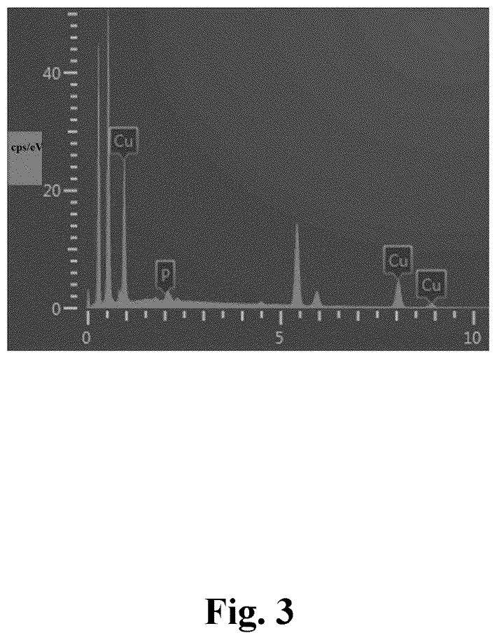

FIG. 4 is the cycle performance pattern of 0.03 wt % BP-Cu-0.3 prepared in Example 1. After five cycles, the catalyst activity does not decrease much, indicating that the catalyst has good cycle stability.

### DETAILED DESCRIPTION

The present invention will be further described in conjunction with specific examples, taking styrene hydrogenation as an example. But the application scope of the present invention is not limited to the raw materials and specific process conditions involved in these examples.

### Example 1

(1) Preparation of dispersion of black phosphorus: 0.4 g of black phosphorus crystal was dispersed in 80 mL of ethanol, and after ultrasonic dispersion for 5 h, the solution obtained was the dispersion of black phosphorus.
(2) Preparation of catalyst: 0.3 g of cetyltrimethyl ammonium bromide was weighed in 20 mL of water (H₂O), and ultrasonic treated for 15 min, into which was then added 0.5 g of tetra-copper trioxide, and stirred for 1 h, into which was then added 10 μL of the dispersion of black phosphorus in the step (1).
(3) 10 μL of phytic acid was added to the step (2), heated in an oil bath at 30° C. for 1 h, and after heating up to 80° C., stirred for 1 h under heating to obtain a uniform mixture.
(4) The mixture obtained in the step (3) was transferred to a hydrothermal reaction kettle equipped with a 90 mL polytetrafluoroethylene lining, and hydrothermally reacted in an oven at 180° C. for 12 h. After the reaction was completed, the reaction kettle was placed in cooling water to be cooled down to terminate the reaction.
(5) The hydrothermally treated catalyst sample obtained in the step (4) was separated into a reaction solution and a required solid by a centrifuge at a rotation speed of 3000 r/min, and the solid obtained was washed alternately with deionized water and absolute ethanol for three times. Finally, the solid was dried in an oven at 60° C. for 20 h.
(6) The solid catalyst sample obtained in the step (5) was placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(7) The obtained catalyst sample calcined in nitrogen atmosphere was placed in a tubular furnace for reduction under a mixed atmosphere containing 90% nitrogen and 10% hydrogen. Before temperature increase, the residual air in the tubular furnace was evacuated by vacuumizing, and then the tube was purged with nitrogen for three times to ensure safety: Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-0.3, where 0.01 wt % was the mass fraction of black phosphorus, and 0.3 was the mass of the surfactant (the same below).

In yet another example of the present invention, the dispersion of black phosphorus was added in the step (2) in an amount of 20 μL, with other steps the same as above, to obtain the product 0.02 wt % BP-Cu-0.3.

In yet another example of the present invention, the dispersion of black phosphorus was added in the step (2) in an amount of 30 μL, with other steps the same as above, to obtain the product 0.03 wt % BP-Cu-0.3.

In yet another example of the present invention, the dispersion of black phosphorus was added in the step (2) in an amount of 40 μL, with other steps the same as above, to obtain the product 0.04 wt % BP-Cu-0.3.

In yet another example of the present invention, the dispersion of black phosphorus was added in the step (2) in an amount of 80 μL, with other steps the same as above, to obtain the product 0.08 wt % BP-Cu-0.3.

In yet another example of the present invention, the dispersion of black phosphorus was added in the step (2) in an amount of 100 μL, with other steps the same as above, to obtain the product 0.10 wt % BP-Cu-0.3.

In yet another example of the present invention, the dispersion of black phosphorus was added in the step (2) in an amount of 200 μL, with other steps the same as above, to obtain the product 0.20 wt % BP-Cu-0.3.

The performance was evaluated by an autoclave evaluation device. The reaction conditions were as follows: 0.1 g of the above catalysts (0.01 wt % BP-Cu-0.3, 0.02 wt % BP-Cu-0.3, 0.03 wt % BP-Cu-0.3, 0:04 wt % BP-Cu-0.3, 0.08 wt % BP-Cu-0.3, 0.10 wt % BP-Cu-0.3 and 0.20 wt % BP-Cu-0.3) were respectively charged into a high-pressure reaction kettle, wherein the hydrogen pressure was 2 MPa, the hydrogenation temperature was 80° C., the concentration of the solution of styrene in ethanol was 30%, and the mass ratio of the metal catalyst to the solution of styrene in ethanol was 1:4. Hydrogen was introduced, and after the reaction kettle was sealed, 0.25 MPa hydrogen was filled into the kettle and then released to replace the air in the kettle. After the process was circulated for five times, hydrogen was charged into the reaction kettle to a required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. The yield of ethylbenzene prepared from styrene is shown in Table 1.

It can be seen from the above experiments that when the dispersion of black phosphorus is continuously added to 200 μL, the catalyst efficiency can substantially achieve a yield of 100%. Therefore, the above technical effect of the present application can be achieved when the addition amount of black phosphorus reaches 1 wt %, 10 wt %, 20 wt %, 30 wt %, 40 wt %, and 50 wt %.

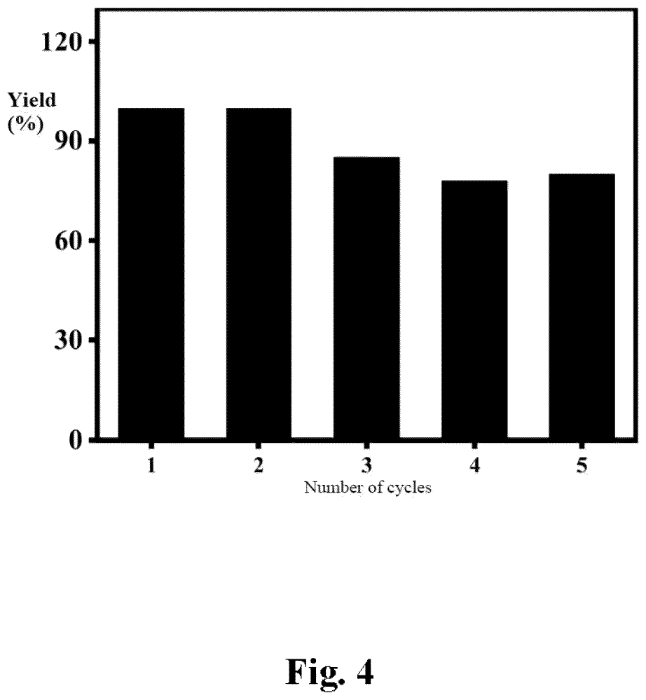

In another case of the present invention, 0.03 wt % BP-Cu-0.3 was selected to carry out the cyclic test of preparing ethylbenzene by catalyzing styrene. The test steps are as follows: after the first catalytic reaction as described above, the separated catalyst was washed with water, then dried, and filled in the high-pressure reaction kettle again to carry out the cyclic catalytic reaction of preparing ethylbenzene from styrene as described above. The yield obtained is shown in FIG. 4.

TABLE 1

| | Catalyst | | | | | | | |
|---|---|---|---|---|---|---|---|---|
| | | 0.01 wt % | 0.02 wt % | 0.03 wt % | 0.04 wt % | 0.08 wt % | 0.1 wt % | 0.2 wt % |
| | Cu | BP-Cu-0.3 | BP-Cu-0.3 | BP-Cu-0.3 | BP-Cu-0.3 | BP-Cu-0.3 | BP-Cu-0.3 | BP-Cu-0.3 |
| Conversion | 10% | 83% | 100% | 100% | 100% | 100% | 100% | 100% |
| ratio | | | | | | | | |

### Example 2

(1) Preparation of dispersion of black phosphorus: 0.4 g of black phosphorus crystal was dispersed in 80 mL of ethanol, and after ultrasonic dispersion for 5 h, the solution obtained was the dispersion of black phosphorus.
(2) Preparation of catalyst: 0.5 g of cetyltrimethyl ammonium bromide was weighed in 20 ml of water (H₂O), and ultrasonic treated for 15 min, into which was then added 0.5 g of tetra-copper trioxide, and stirred for 1 h, into which was then added 10 μL of the dispersion of black phosphorus in the step (1).
(3) 10 μL of phytic acid was added to the step (2), heated in an oil bath at 30° C. for 1 h, and after heating up to 80° C., stirred for 1 h under heating to obtain a uniform mixture.
(4) The mixture obtained in the step (3) was transferred to a hydrothermal reaction kettle equipped with a 90 mL polytetrafluoroethylene lining, and continued to react in an oven at 180° C. for 12 h. After the reaction was completed, the reaction kettle was placed under cold water flow to be quickly cooled down to terminate the reaction.
(5) The hydrothermally treated catalyst sample obtained in the step (4) was separated into a reaction solution and a required solid by a centrifuge at a rotation speed of 3000 r/min, and the solid obtained was washed alternately with deionized water and absolute ethanol for three times. Finally, the solid was dried in an oven at 60° C. for 20 h.
(6) The solid catalyst sample obtained in the step (5) was placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(7) The obtained catalyst sample calcined in nitrogen atmosphere was placed in a tubular furnace for reduction under a mixed atmosphere containing 90% nitrogen and 10% hydrogen. Before temperature increase, the residual air in the tubular furnace was evacuated by vacuumizing, and then the tube was purged with nitrogen for three times to ensure safety. Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-0.5, where 0.01 wt % was the mass fraction of black phosphorus, and 0.5 was the mass of the surfactant (the same below).

In yet another example of the present invention, the amount of cetyltrimethyl ammonium bromide added in the step (2) was 0.7, with other steps the same as above, to obtain the product 0.01 wt % BP-Cu-0.7.

In yet another example of the present invention, the amount of cetyltrimethyl ammonium bromide added in the step (2) was 1.5, with other steps the same as above, to obtain the product 0.01 wt % BP-Cu-1.5.

In yet another example of the present invention, the amount of cetyltrimethyl ammonium bromide added in the step (2) was 3.0, with other steps the same as above, to obtain the product 0.01 wt % BP-Cu-3.0.

In yet another example of the present invention, the amount of cetyltrimethyl ammonium bromide added in the step (2) was 5.0, with other steps the same as above, to obtain the product 0.01 wt % BP-Cu-5.0.

In yet another example of the present invention, the amount of cetyltrimethyl ammonium bromide added in the step (2) was 7.0, with other steps the same as above, to obtain the product 0.01 wt % BP-Cu-7.0.

In yet another example of the present invention, the amount of cetyltrimethyl ammonium bromide added in the step (2) was 10.0, with other steps the same as above, to obtain the product 0.01 wt % BP-Cu-10.0.

The performance was evaluated by an autoclave evaluation device. The reaction conditions were as follows: 0.1 g of the above catalysts (0.01 wt % BP-Cu-0.5, 0.01 wt % BP-Cu-0.7, 0.01 wt % BP-Cu-1.5, 0.01 wt % BP-Cu-3.0, 0.01 wt % BP-Cu-5.0, 0.01 wt % BP-Cu-7.0 and 0.01 wt % BP-Cu-10.0) were respectively charged into a high-pressure reaction kettle, wherein the hydrogen pressure was 2 MPa, the hydrogenation temperature was 80° C., the concentration of the solution of styrene was 30%, and the mass ratio of the metal catalyst to the solution of styrene in ethanol was 1:4. Hydrogen was introduced, and after the reaction kettle was sealed, 0.25 MPa hydrogen was filled into the kettle and then released to replace the air in the kettle. After the process was circulated for five times, hydrogen was charged into the reaction kettle to a required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. The conversion ratio of ethylbenzene prepared from styrene is shown in Table 2.

TABLE 2

| | Catalyst | | | | | | |
|---|---|---|---|---|---|---|---|
| | 0.01 wt % | 0.01 wt % | 0.01 wt % | 0.01 wt % | 0.01 wt % | 0.01 wt % | 0.01 wt % |
| | BP-Cu-0.5 | BP-Cu-0.7 | BP-Cu-1.5 | BP-Cu-3.0 | BP-Cu-5.0 | BP-Cu-7.0 | BP-Cu-10.0 |
| Conversion | 90% | 98% | 100% | 100% | 100% | 100% | 100% |
| ratio | | | | | | | |

### Example 3

(1) Preparation of dispersion of black phosphorus: 0.4 g of black phosphorus crystal was dispersed in 80 mL of ethanol, and after ultrasonic dispersion for 5 h, the solution obtained was the dispersion of black phosphorus.
(2) Preparation of catalyst: 0.7 g of cetyltriethylammonium bromide was weighed in 20 ml of water (H₂O), and ultrasonic treated for 15 min, into which was then added 0.5 g of tetra-copper trioxide, and stirred for 1 h, into which was then added 10 μL of the dispersion of black phosphorus in the step (1).
(3) 10 μL of phytic acid was added to the step (2), heated in an oil bath at 30° C. for 1 h, and after heating up to 80° C., stirred for 1 h under heating to obtain a uniform mixture.
(4) The mixture obtained in the step (3) was transferred to a hydrothermal reaction kettle equipped with a 90 mL polytetrafluoroethylene lining, and continued to react in an oven at 180° C. for 12 h. After the reaction was completed, the reaction kettle was placed under cold water flow to be quickly cooled down to terminate the reaction.
(5) The hydrothermally treated catalyst sample obtained in the step (4) was separated into a reaction solution and a required solid by a centrifuge at a rotation speed of 3000 r/min, and the solid obtained was washed alternately with deionized water and absolute ethanol for three times. Finally, the solid was dried in an oven at 60° C. for 20 h.
(6) The solid catalyst sample obtained in the step (5) was placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(7) The obtained catalyst sample calcined in nitrogen atmosphere was placed in a tubular furnace for reduction under a mixed atmosphere containing 90% nitrogen and 10% hydrogen. Before temperature increase, the residual air in the tubular furnace was evacuated by vacuumizing, and then the tube was purged with nitrogen for three times to ensure safety. Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-0.7-1.

In another embodiment of this example, the surfactant was cetyltriethylammonium chloride, to obtain the product 0.01 wt % BP-Cu-0.7-2.

In another embodiment of this example, the surfactant was octadecyl trimethyl ammonium bromide, to obtain the product 0.01 wt % BP-Cu-0.7-3.

In another embodiment of this example, the surfactant was Pluronic F127, to obtain the product 0.01 wt % BP-Cu-0.7-4.

In another embodiment of this example, the surfactant was polyvinylpyrrolidone, to obtain the product 0.01 wt % BP-Cu-0.7-5.

In another embodiment of this example, the raw material of black phosphorus was a black phosphorus quantum dot, to obtain the product 0.01 wt % BP-Cu-0.7-6.

In another embodiment of this example, the copper catalyst was elemental copper, to obtain the product 0.01 wt % BP-Cu-0.7-7.

In another embodiment of this example, the copper catalyst was copper oxide, to obtain the product 0.01 wt % BP-Cu-0.7-8.

In another embodiment of this example, the copper catalyst was cuprous oxide, to obtain the product 0.01 wt % BP-Cu-0.7-9.

The performance was evaluated by an autoclave evaluation device. The reaction conditions were as follows: 0.1 g of the catalysts prepared as described above (0.01 wt % BP-Cu-0.7-1, 0.01 wt % BP-Cu-0.7-2, 0.01 wt % BP-Cu-0.7-3, 0.01 wt % BP-Cu-0.7-4, 0.01 wt % BP-Cu-0.7-5, 0.01 wt % BP-Cu-0.7-6, 0.01 wt % BP-Cu-0.7-7, 0.01 wt % BP-Cu-0.7-8 and 0.01 wt % BP-Cu-0.7-9) were respectively charged into a high-pressure reaction kettle, wherein the hydrogen pressure was 2 MPa, the hydrogenation temperature was 80° C., the concentration of the solution of styrene was 30%, and the mass ratio of the metal catalyst to styrene was 1:4. Hydrogen was introduced into the reaction kettle, and after the reaction kettle was sealed, 0.25 MPa hydrogen was filled into the kettle and then released to replace the air in the kettle. After the process was circulated for five times, hydrogen was charged into the reaction kettle to the required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. Conversion ratio is as shown in Table 3.

TABLE 3

| | Catalyst | | | | | | | | |
|---|---|---|---|---|---|---|---|---|---|
| | 0.01 wt % | wt % | 0.01 wt % | 0.01 wt % | 0.01 wt % | 0.01 wt % | 0.01 wt % | 0.01 wt % | 0.01 wt % |
| | BP-Cu-0.7-1 | BP-Cu-0.7-2 | BP-Cu-0.7-3 | BP-Cu-0.7-4 | BP-Cu-0.7-5 | BP-Cu-0.7-6 | BP-Cu-0.7-7 | BP-Cu-0.7-8 | BP-Cu-0.7-9 |
| Conversion | 83% | 89% | 100% | 100% | 100% | 100% | 80% | 98% | 98% |
| ratio | | | | | | | | | |

### Example 4

(1) Preparation of dispersion of black phosphorus: 0.4 g of black phosphorus crystal was dispersed in 80 mL of ethanol, and after ultrasonic dispersion for 5 h, the solution obtained was the dispersion of black phosphorus.
(2) Preparation of catalyst: 1.0 g of cetyltrimethyl ammonium bromide was weighed in 20 mL of water (H₂O), and ultrasonic treated for 15 min, into which was then added 0.5 g of tetra-copper trioxide, and stirred for 1 h, into which was then added 10 μL of the dispersion of black phosphorus in the step (1).
(3) The mixture obtained in the step (2) was heated in an oil bath at 30° C. for 1 h, and after heating up to 80° C., stirred for 1 h under heating to obtain a uniform mixture.
(4) The mixture obtained in the step (3) was transferred to a hydrothermal reaction kettle equipped with a 90 mL polytetrafluoroethylene lining, and continued to react in an oven at 180° C. for 12 h. After the reaction was completed, the reaction kettle was placed under cold water flow to be quickly cooled down to terminate the reaction.
(5) The hydrothermally treated catalyst sample obtained in the step (4) was separated into a reaction solution and a required solid by a centrifuge at a rotation speed of 3000 r/min, and the solid obtained was washed alternately with deionized water and absolute ethanol for three times. Finally, the solid was dried in an oven at 60° C. for 20 h.
(6) The solid catalyst sample obtained in the step (5) was placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(7) The obtained catalyst sample calcined in nitrogen atmosphere was placed in a tubular furnace for reduction under a mixed atmosphere containing 90% nitrogen and 10% hydrogen. Before temperature increase, the residual air in the tubular furnace was evacuated by vacuumizing, and then the tube was purged with nitrogen for three times to ensure safety. Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-1.0-1.

The performance was evaluated by an autoclave evaluation device. The reaction conditions were as follows: 0.1 g of the catalyst 0.01 wt % BP-Cu-1.0-1 was charged into a high-pressure reaction kettle, wherein the hydrogen pressure was 2 MPa, the hydrogenation temperature was 80° C., the concentration of the solution of styrene was 30%, and the mass ratio of the metal catalyst to styrene was 1:4. Hydrogen was introduced into the reaction kettle, and after the reaction kettle was sealed, 0.25 MPa hydrogen was filled into the kettle and then released to replace the air in the kettle. After the process was circulated for five times, hydrogen was charged into the reaction kettle to the required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. The conversion ratio was 92%.

### Example 5

(1) Preparation of dispersion of black phosphorus: 0.4 g of black phosphorus crystal was dispersed in 80 mL of N-methylpyrrolidone, and after ultrasonic dispersion for 5 h, the solution obtained was the dispersion of black phosphorus.
(2) Preparation of catalyst: 0.5 g of tetra-copper trioxide was weighed in 20 mL of water (H₂O), ultrasonic treated for 15 min, and stirred for 1 h, into which was then added 10 μL of the dispersion of black phosphorus in the step (1). (3) The mixture obtained in the step (2) was heated in an oil bath at 30° C. for 1 h, and after heating up to 80° C., stirred for 1 h under heating to obtain a uniform mixture.
(4) The mixture obtained in the step (3) was transferred to a hydrothermal reaction kettle equipped with a 90 mL polytetrafluoroethylene lining, and continued to react in an oven at 180° C. for 12 h. After the reaction was completed, the reaction kettle was placed under cold water flow to quickly cool down to terminate the reaction.
(5) The hydrothermally treated catalyst sample obtained in the step (4) was separated into a reaction solution and a required solid by a centrifuge at a rotation speed of 3000 r/min, and the solid obtained was washed alternately with deionized water and absolute ethanol for three times. Finally, the solid was dried in an oven at 60° C. for 20 h.
(6) The solid catalyst sample obtained in the step (5) was placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(7) The obtained catalyst sample calcined in nitrogen atmosphere was placed in a tubular furnace for reduction under a mixed atmosphere containing 90% nitrogen and 10% hydrogen. Before temperature increase, the residual air in the tubular furnace was evacuated by vacuumizing, and then the tube was purged with nitrogen for three times to ensure safety. Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-1.

The performance was evaluated by an autoclave evaluation device. The reaction conditions were as follows: 0.1 g of the catalyst 0.01 wt % BP-Cu-1 was charged into a high-pressure reaction kettle, wherein the hydrogen pressure was 2 MPa, the hydrogenation temperature was 80° C., the concentration of the solution of styrene was 30%, and the mass ratio of the metal catalyst to styrene was 1:4. Hydrogen was introduced into the reaction kettle, and after the reaction kettle was sealed, 0.25 MPa hydrogen was filled into the kettle and then released to replace the air in the kettle. After the process was circulated for five times, hydrogen was charged into the reaction kettle to the required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. The conversion ratio was 92%.

### Example 6

(1) Tetra-copper trioxide powder was taken, into which were added black phosphorus crystals, stirred well, and then poured into an agate ball milling tank. Dimethyl sulfoxide was added for wetting, and then grinding balls were added, and the mixture was ball milled at a rotation speed of 800 r/min for 12 h. During ball milling, cold air of 15° C. was introduced to prevent black phosphorus oxidation caused by temperature increase during ball milling. The particle size of the black phosphorus powder was below 120 meshes, the mass ratio of the black phosphorus crystal to tetra-copper trioxide was 1:10000, the material of the grinding ball was agate, the diameter was 2 cm, the volume of the ball milling tank was 100 mL, and the mass ratio of the copper-based catalyst to the grinding ball was 1:500.
(2) The solid catalyst sample obtained in the step (1) was sieved to 800 meshes and more, and then placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(3) The obtained catalyst sample calcined in nitrogen atmosphere was placed in a tubular furnace for reduction under a mixed atmosphere containing 90% nitrogen and 10% hydrogen. Before temperature increase, the residual air in the tubular furnace was evacuated by vacuumizing, and then the tube was purged with nitrogen for three times to ensure safety. Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-1.

The performance was evaluated by an autoclave evaluation device. The reaction conditions were as follows: 0.1 g of the catalyst 0.01 wt % BP-Cu-1 was charged into a high-pressure reaction kettle, wherein the hydrogen pressure was 2 MPa, the hydrogenation temperature was 80° C., the concentration of the solution of styrene was 30%, and the mass ratio of the metal catalyst to styrene was 1:4. Hydrogen was introduced into the reaction kettle, and after the reaction kettle was sealed, 0.25 MPa hydrogen was filled into the kettle and then released to replace the air in the kettle. After the process was circulated for five times, hydrogen was charged into the reaction kettle to the required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. The conversion ratio was 90%.

### Example 7

(1) Preparation of dispersion of black phosphorus: 0.4 g of black phosphorus crystal was dispersed in 80 mL of N,N-dimethylacetamide, and after ultrasonic dispersion for 5 h, the solution obtained was the dispersion of black phosphorus.
(2) Preparation of catalyst: 0.3 g of sodium dodecyl benzene sulfonate was weighed in 20 mL of water (H₂O), and ultrasonic treated for 15 min, into which was then added 0.5 g of tetra-copper trioxide, and stirred for 1 h, into which was then added 10 μL of the dispersion of black phosphorus in the step (1).
(3) 10 μL of phytic acid was added to the step (2), heated in an oil bath at 30° C. for 1 h, and after heating up to 80° C., stirred for 1 h under heating to obtain a uniform mixture.
(4) The hydrothermally treated catalyst sample obtained in the step (3) was separated into a reaction solution and a required solid by a centrifuge at a rotation speed of 3000 r/min, and the solid obtained was washed alternately with deionized water and absolute ethanol for three times. Finally, the solid was dried in an oven at 60° C. for 20 h.
(5) The solid catalyst sample obtained in the step (4) was placed in a tubular furnace for calcination under nitrogen atmosphere, wherein the calcination temperature was raised to 200° C. at a ramp rate of 5° C./min, and then kept for 5 h, with a nitrogen flow rate kept at 110 mL/min during the temperature increase, maintenance and decrease. After the temperature was decreased to room temperature, the catalyst sample obtained was taken out.
(6) The obtained catalyst sample calcined in nitrogen atmosphere was placed in a tubular furnace for reduction under a mixed atmosphere containing 95% nitrogen and 5% hydrogen. Before temperature increase, the residual air in the tubular furnace was evacuated by vacuumizing, and then the tube was purged with nitrogen for three times to ensure safety. Subsequently, the ramp rate of the tubular furnace was set at 5° C./min, and the temperature was raised to 200° C., and then kept for 4 h. After the reaction was completed and the temperature was decreased to room temperature, the sample was taken out. The resulting final catalyst sample was taken out and recorded as 0.01 wt % BP-Cu-0.3-1.

The performance was evaluated by an autoclave evaluation device. The reaction conditions were as follows: 0.1 g of the catalyst 0.01 wt % BP-Cu-0.3-1 was charged into a high-pressure reaction kettle, wherein the hydrogen pressure was 2 MPa, the hydrogenation temperature was 80° C., the concentration of the solution of styrene was 30%, and the mass ratio of the metal catalyst to styrene was 1:4. Hydrogen was introduced into the reaction kettle, and after the reaction kettle was sealed, 0.25 MPa hydrogen was filled into the kettle and then released to replace the air in the kettle. After the process was circulated for five times, hydrogen was charged into the reaction kettle to the required pressure (2 MPa), with a reaction temperature of 60° C. and a reaction time of 2 h. After the reaction was completed, the reaction kettle was cooled to room temperature. Finally, the reaction liquid was separated from the catalyst by an operation such as filtration and centrifugation. The conversion ratio was 90%.

### Example 8

The catalyst 0.02 wt % BP-Cu-0.3 prepared in Example 1 was used to carry out the test of preparing ethylbenzene by catalyzing phenylacetylene.

The steps of the catalytic reaction were the same as in Example 1. The conversion ratio was 100%.

### Example 9

The catalyst 0.02 wt % BP-Cu-0.3 prepared in Example 1 was used to carry out the test of preparing tetrahydroquinoline by catalyzing quinoline.

The reaction conditions of the catalytic reaction were: Pressure: 2 MPa, Temperature: 120° C., and other steps were the same as in Example 1. The conversion ratio was 80%.

### Example 10

The catalyst 0.02 wt % BP-Cu-0.3 prepared in Example 1 was used to carry out the test of preparing methyl glycolate by catalytic hydrogenation of dimethyl oxalate.

The reaction conditions of the catalytic reaction were: 0.2 g of the catalyst, 4 MPa, 180° C., and other steps were the same as in Example 1. The conversion ratio was 89%.

### Example 11

The catalyst 0.01 wt % BP-Cu-1 prepared in Example 5 was used to carry out the test of preparing furfuryl alcohol by catalytic hydrogenation of furfural.

The reaction conditions of the catalytic reaction were: 0.1 g of the catalyst, 2 MPa, 120° C., and other steps were the same as in Example 5. The selectivity of furfuryl alcohol was 99%.

### Example 12

The catalyst 0.01 wt % BP-Cu-1 prepared in Example 6 was used to carry out the test of preparing p-chloroaniline by catalytic hydrogenation of p-chloronitrobenzene.

The reaction conditions of the catalytic reaction were: 0.5 g of the catalyst, 1 MPa, 80° C., and other steps were the same as in Example 6. The selectivity of p-chloroaniline was 90%.

### Example 13

The catalyst 0.01 wt % BP-Cu-1 prepared in Example 6 was used to carry out the test of preparing ethanol by catalyzing ethyl acetate.

The reaction conditions of the catalytic reaction were: 0.1 g of the catalyst, 2 MPa, 250° C., and other steps were the same as in Example 6. The selectivity of ethanol was 95%

1. A black phosphorus-modified copper-based catalyst, wherein, the copper-based catalyst is selected from the group consisting of elemental copper, copper oxide, cuprous oxide, or tetra-copper trioxide; and in the black phosphorus-modified copper-based catalyst, a mass fraction of black phosphorus is 0.001 wt %-50 wt %.

2. The black phosphorus-modified copper-based catalyst according to claim 1, wherein a raw material of black phosphorus in the black phosphorus-modified copper-based catalyst is selected from one or more of the group consisting of black phosphorus powder, black phosphorus quantum dots, black phosphorus nanosheets and black phosphorus crystals.

3. The black phosphorus-modified copper-based catalyst according to claim 2, wherein the raw material of black phosphorus in the black phosphorus-modified copper-based catalyst is selected from the black phosphorus crystals; and a mass fraction of the black phosphorus is 0.01-50 wt %.

4. The black phosphorus-modified copper-based catalyst according to claim 3, wherein the mass fraction of the black phosphorus is selected from any one of the group consisting of 0.01 wt %, 0.02 wt %, 0.03 wt %, 0.04 wt %, 0.05 wt %, 0.06 wt %, 0.07 wt %, 0.08 wt %, 0.09 wt %, 0.10 wt %, 0.20 wt %, 1 wt %, 10 wt %, 20 wt %, 30 wt %, 40 wt %, and 50 wt %.

5. A method for preparing the black phosphorus-modified copper-based catalyst according to claim 1, comprising the following steps: (1) adding a raw material of black phosphorus into a copper-based catalyst to form a mixture; (2) allowing the mixture to react; and (3) further separating a product obtained after the reaction is completed, drying, roasting and reducing the product under hydrogen, to obtain the black phosphorus-modified copper-based catalyst.

6. The method for preparing the black phosphorus-modified copper-based catalyst according to claim 5, wherein the reaction in the step (2) is one or more of a hydrothermal reaction, a stirring reflux reaction, or ball milling.

7. The method for preparing the black phosphorus-modified copper-based catalyst according to claim 5, wherein
in the hydrothermal reaction, the mixture formed by adding the raw material of black phosphorus into the copper-based catalyst is transferred to a polytetrafluoroethylene lining at a hydrothermal temperature of 120-200° C. for 1-8 h;
in the stirring reflux reaction, the mixture formed by adding the raw material of black phosphorus into the copper-based catalyst is transferred to a reactor, and reacted under reflux for 0.1-10 h at a temperature of 100-200° C. and a stirring speed of 50-1000 r/min under stirring;
in the ball milling, the mixture formed by adding the raw material of black phosphorus into the copper-based catalyst is ball milled for 1-15 h at a rotating speed of 800-1200 r/min.

8. The method for preparing the black phosphorus-modified copper-based catalyst according to claim 7, wherein during the hydrothermal reaction and the stirring reflux reaction, the raw material of black phosphorus is first dispersed in a solvent selected from one or more of the group consisting of: absolute ethanol, N-methylpyrrolidone, N-vinylpyrrolidone, N-cycloethylpyrrolidone, N-octylpyrrolidone, formamide, N-methylformamide, N,N-dimethylformamide, N,N-dimethylacetamide, dimethyl sulfoxide, methanol, ethanol, ethylene glycol, isopropanol, tert-butanol, acetone, 2-pentanone and water, to form a solution of black phosphorus;
during the ball milling, the raw material of black phosphorus is wetted by adding the solvent and then ball milled.

9. The method for preparing the black phosphorus-modified copper-based catalyst according to claim 7, wherein during the hydrothermal reaction, the stirring reflux reaction or the ball milling, a surfactant is selectively added, and the surfactant is selected from one or more of the group consisting of cetyltrimethyl ammonium bromide, cetyltrimethyl ammonium chloride, cetyltriethylammonium bromide, octadecyl dimethyl ammonium chloride, octadecyl trimethyl ammonium bromide, Pluronic F127, polyvinylpyrrolidone, sodium dodecyl sulfate and sodium dodecyl benzene sulfonate, and has a mass fraction of 0.1 wt %-10 wt %.

10. The method for preparing the black phosphorus-modified copper-based catalyst according to claim 9, wherein a mass fraction of the surfactant cetyltrimethyl ammonium bromide is 0.1 wt %-5 wt %.

11. The method for preparing the black phosphorus-modified copper-based catalyst according to claim 10, wherein the mass fraction of the surfactant is selected from any one of the group consisting of 0.3 wt %, 0.5 wt %, 1.0 wt %, 1.5 wt %, 2.0 wt %, 2.5 wt %, 3.0 wt %, 3.5 wt %, 4.0 wt %, 4.5 wt %, and 5.0 wt %.

12. The method for preparing the black phosphorus-modified copper-based catalyst according to claim 7, wherein phytic acid is selectively added into a mixed solution before the hydrothermal reaction, the stirring reflux reaction or the ball milling, and stirred and mixed well after being heating up to 80-100° C. under an oil bath, and an addition amount of the phytic acid is 0.1-5 wt % of the copper-based catalyst by mass.

13. The method for preparing the black phosphorus-modified copper-based catalyst according to claim 5, wherein the separation comprises any one of centrifugation, sieving and filtration.

14. The method for preparing the black phosphorus-modified copper-based catalyst according to claim 5, wherein
the roasting has a temperature of 200-700° C., and an inert atmosphere used during the roasting is selected from any one of the group consisting of argon, nitrogen or helium.

15. The method for preparing the black phosphorus-modified copper-based catalyst according to claim 5, wherein
the reduction under hydrogen refers to a reduction reaction conducted after heating up to 160-450° C. under a mixed atmosphere of hydrogen and nitrogen, and the product obtained after the reduction reaction is the black phosphorus-modified copper-based catalyst; and in the mixed atmosphere of hydrogen and nitrogen, a volume fraction of hydrogen is 5-10%.

16. A method of the black phosphorus-modified copper-based catalyst according to claim 1 in a hydrogenation reduction reaction of an organic compound, comprising the following steps:
transferring a solution of an organic compound to a lining of an autoclave, adding the black phosphorus-modified copper catalyst, introducing hydrogen in a closed environment, continuously introducing hydrogen after air is replaced by hydrogen, and reacting for 0.5-5 h at a pressure of 0.5-10 MPa and a temperature of 60° C.-200° C. to obtain a reduction product.

17. The method according to claim 16, wherein a mass ratio of the catalyst to the organic compound is 1:3-10.

18. The method according to claim 17, wherein a solvent used in the solution of the organic compound is selected from the group consisting of C1-C4 alcohol solvents, and a mass concentration of the organic compound is 10-50%.

19. The method according to claim 18, wherein the organic compound comprises any one of styrene, phenylacetylene, quinoline, furfural, dimethyl oxalate, nitrobenzene, p-chloronitrobenzene, p-nitrophenol or ethyl acetate.

20. A method of the black phosphorus-modified copper-based catalyst prepared according to claim 4 in a hydrogenation reduction reaction of an organic compound, comprising the following steps:
transferring a solution of an organic compound to a lining of an autoclave, adding the black phosphorus-modified copper catalyst, introducing hydrogen in a closed environment, continuously introducing hydrogen after air is replaced by hydrogen, and reacting for 0.5-5 h at a pressure of 0.5-10 MPa and a temperature of 60° C.-200° C. to obtain a reduction product.
